(19) United States
(12) Patent Application Publication
(10) Pub. No.: US 2016/0081741 A1
(43) Pub. Date: Mar. 24, 2016

(54) ABLATION APPARATUS

(71) Applicant: Angel Medical Technology (Nanjing) Co., Ltd., Nanjing City (CN)

(72) Inventor: Chi Ming Chan, Guangzhou (CN)

(21) Appl. No.: 14/711,953

(22) Filed: May 14, 2015

(30) Foreign Application Priority Data
Sep. 18, 2014 (CN) 201410480241.0

(51) Int. Cl. A61B 18/14

(52) U.S. Cl. CPC A61B 18/1477; A61B 2018/00083

(57) ABSTRACT

An ablation needle is disclosed as including a needle body (1) and a needle head (2, 9), and a positive temperature coefficient (PTC) sleeve (7, 10) in a heat-transferrable relationship with the needle body (1).

### BACKGROUND

1. Technical Field

This invention relates to the field of medical instruments, in particular an ablation device.

2. Discussion of Related Art

Radio frequency ablation (RFA) is a minimally invasive treatment method. Rossi, McGahan et al. first reported in 1990 the use of RFA in ablation of liver tissues of animals. This technique was subsequently used in treatment of human hepatic tumors. Nowadays, RFA technique is widely used in treatment of diseases of various organs in the body. In addition to inactivating tumors, it can also lower tumor load, thus reducing pain and hormone secretion. There are also studies of its use in treating non-neoplastic diseases, such as hypersplenism. Basically, RFA is a kind of thermotherapy on tumors. Its basic principle is the use of heat energy to damage tumor tissues. Radio frequency waves generated by electrodes causes the ions and polar macro-molecules in the surrounding tissues to vibrate, impact on one another and rub against one another, thus generating heat. The tumor region is heated up to an effective treatment temperature range and maintained for a period of time, so as to kill the tumor cells. At the same time, the radio frequency heat effect can realize intravascular coagulation of the surrounding tissues, thus forming a reaction zone, and occluding blood supply to the tumors, thus preventing tumor metastasis.

Regarding the control of the extent of ablation, there are currently two methods: impedance control and temperature control.

An electrical resistance of 400-500Ω is usually used in impedance control, but the exact resistance cannot be easily adjusted. Too low a resistance will cause premature ending of ablation and thus incomplete ablation, whereas too high a resistance will easily bring about adhesion of tissues.

In temperature control, the threshold temperature value is usually set at 125° C., and can easily cause an uneven degree of ablation in parts of the treatment region.

### SUMMARY

It is thus an object of the present invention to provide an ablation device in which the aforesaid shortcomings are mitigated or at least to provide a useful alternative to the trade and public.

According to the present invention, there is provided an ablation device including a needle with a needle body and a needle head, and at least one positive temperature coefficient (PTC) member in a heat-transferrable relationship with said needle body.

### BRIEF DESCRIPTION OF THE DRAWINGS

Ablation devices according to the present invention will now be described, by way of examples only, with reference to the accompany drawings, in which:

FIG. 1 is a longitudinal sectional view of a bipolar ablation needle according to a first embodiment of the present invention,

FIG. 2 is an enlarged view of the encircled part marked I in FIG. 1, and

FIG. 3 is a partial longitudinal sectional view of a unipolar ablation needle according to a second embodiment of the present invention.

### DETAILED DESCRIPTION

A Positive Temperature Coefficient (PTC) thermistor is a semiconductor having electrical resistance with good temperature sensitivity. The term “PTC” is usually used for referring to semiconductor devices, parts or components with very large positive temperature coefficients. When exceeding a certain temperature, its electrical resistance will increase step-wise when the temperature increases. The higher the temperature, the higher the electrical resistance.

The present invention makes use of the characteristics of PTC thermistors to provide a new ablation device.

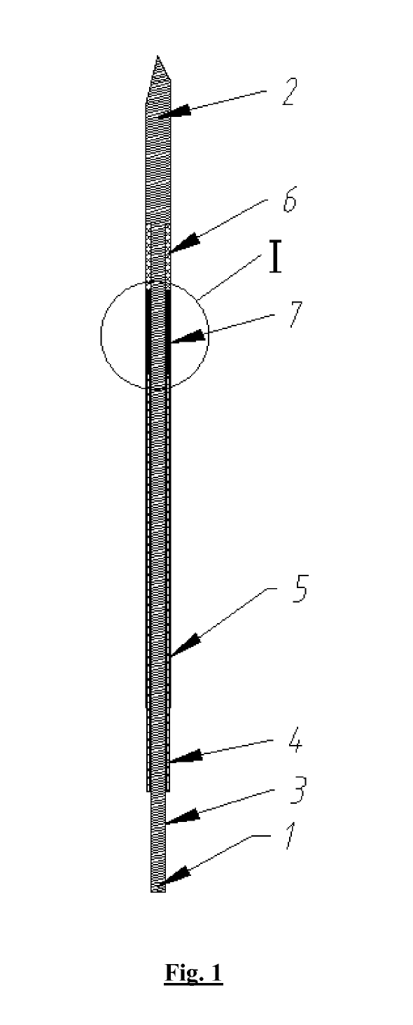

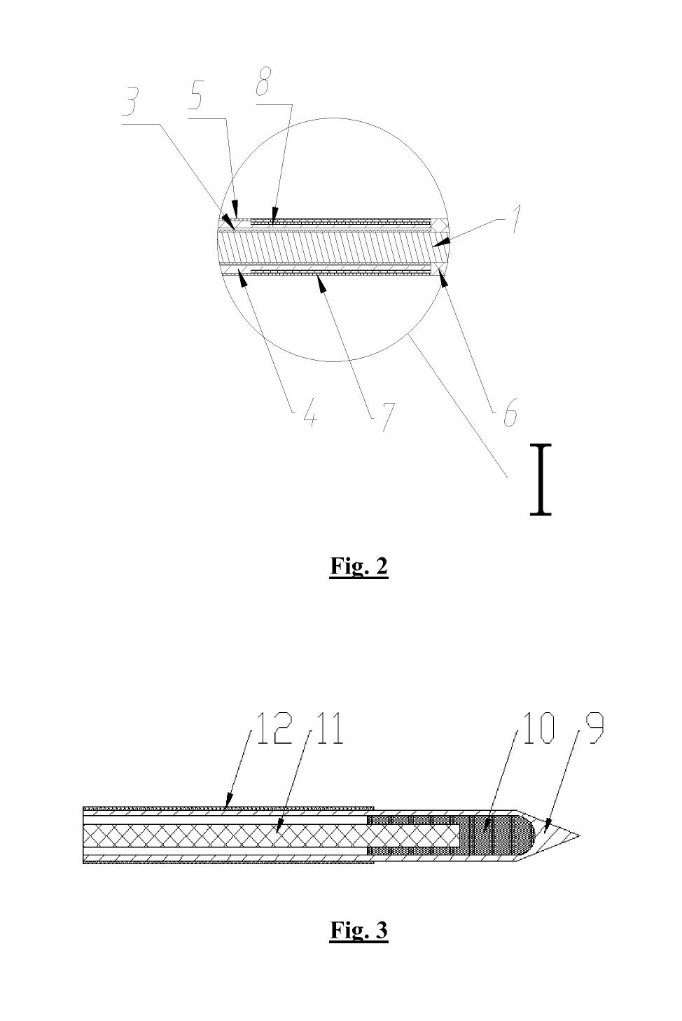

As shown in FIGS. 1 and 2, a bipolar ablation needle, being an ablation device according to a first embodiment of the present invention, includes a solid needle body 1 and a needle head 2 with a pointed end. The device further includes an inner electrically insulating sleeve 3 surrounding and in contact with part of an outer cylindrical surface of the needle body 1, an electrode sleeve 4 surrounding and in contact with part of an outer cylindrical surface of the inner insulating sleeve 3, an outer electrically insulating sleeve 5 surrounding and in contact with part of an outer cylindrical surface of the electrode sleeve 4, an isolating electrically insulating layer 6 surrounding and in contact with part of an outer cylindrical surface of the needle body 1, and a PTC sleeve 7 surrounding and in contact with part of an outer cylindrical surface of the electrode sleeve 4. The PTC sleeve 7 is electrically connected with the electrode sleeve 4, and heat generated by the PTC sleeve 7 may be transferred to the needle body 1 of the ablation needle.

The needle head 2 and the needle body 1 are integral with each other. The needle head 2 is at one longitudinal end of the needle body 1, of a diameter larger than that of the needle body 1, and is sharp. The isolating insulating layer 6 is positioned on the body 1 and next to the needle head 2. Adjacent a longitudinal end of the isolating insulating layer 6 away from the needle head 2 are provided, starting from the inner most layer, the inner insulating sleeve 3, the electrode sleeve 4, the PTC sleeve 7, and the outer insulating sleeve 5.

A stepped portion 8 is formed adjacent a longitudinal end of the isolating insulating layer 6 away from the needle head 2. The stepped portion 8 is of a diameter smaller than the largest diameter of the electrode sleeve 4. The PTC sleeve 7 is set inside the stepped portion 8. It can be seen that, by way of such an arrangement, the PTC sleeve 7, though not in direct contact with the needle body 1, surrounds the needle body 1, such that heat generated by the PTC sleeve 7 may be transferred to the needle body 1.

The isolating insulating layer 6 may be made of polytetrafluoroethylene (PTFE). Both the needle body 1 and the needle head 2 may be made of medical-grade 304 stainless steel. Each of the inner insulating sleeve 3 and/or the outer insulating sleeve 5 may be made of a PTFE-based polymer traded under the trade mark TEFLON®. The pointed end of the needle head 2 may be of an angle of between 10° to 20°, and preferably of 14.5°.

As shown in FIG. 3, a unipolar ablation needle, being an ablation device according to a second embodiment of the present invention, includes an ablation needle with a hollow needle body and a needle head 9 with a pointed end. A PTC sleeve 10 is received within the interior of the hollow needle body and directly contacts the needle body, such that heat generated by the PTC sleeve 10 may be transferred to the needle body. The PTC sleeve 10 is also partly hollow to receive part of an electrode 11 for establishing electrical contact there-between. An outer insulating sleeve 12 surrounds and is in contact with part of an outer cylindrical surface of the needle body.

Both the needle body and the needle head 9 (which are integral with each other) of the unipolar ablation needle may be made of medical-grade 304 stainless steel. The outer insulating sleeve 12 may be made of a PTFE-based polymer traded under the trade mark TEFLON®. The pointed end of the needle head 9 may be of an angle of between 10° to 20°, and preferably of 14.5°.

The PTC sleeves 7, 10 are distributed with PTC thermistor/material, which may be made of:
(a) nylon-12 (L1940), namely a nylon polymer with the formula [(CH₂)₁₁C(O)NH]ₙ manufactured by Degussa AG (Germany),
(b) superconducting carbon black (with an oil absorption value of 780 cm³/100 g) manufactured by Shandong Zibo Carbon Black Factory, and
(c) fumed silica (R106) manufactured by China BlueStar Shenyang Chemical Co. Ltd.

Dried and processed nylon-12, carbon black and fumed silica are mixed and processed in a torque rheometer at a temperature of around 190° C. for about 10 minutes. The thus mixed and processed materials are then conveyed to a mould at a temperature of around 200° C. to form PTC sleeves. The pressure is maintained for 2 minutes and the PTC sleeves are allowed to cool down naturally under room temperature. The resultant sleeves with PTC material/thermistor have an electrical resistance of 1.45×10⁻³Ω at room temperature, and with an electrical resistivity of 1.1×10⁻²Ω cm. The electrical resistance of such PTC sleeves change drastically at around 100° C., with a change of a magnitude of around 1×10⁹. Such PTC sleeves thus have a low electrical resistance at room temperature and a large rate of change of electrical resistance.

The ablation device according to the present invention makes use of the characteristics of PTC thermistor. In combination with a radio frequency generator, such a device may be specifically used for heat treatment of target biological tissues. Such heat treatment may be carried out within the temperature range of 80° C. to 120° C., and preferably 100° C., such that upon coagulation of the tissues, there is no adhesion of the ablation devices, thus reducing the risk of such complications as bleeding.

As the PTC thermistor in the present invention is distributed about the entire ablation region, in contrast to existing total cutting-through, an ablation device according to the present invention can achieve partial cutting-through, until all the tissues in the ablation region reach the predetermined ablation temperature, thus realizing precise control of the ablation process, and total and complete ablation of all target tissues. In addition, once the target ablation tissues reach the predetermined temperature, the tissues will be cut, thus avoiding over damage to the tissues.

It should be understood that the above only illustrates examples whereby the present invention may be carried out, and that various modifications and/or alterations may be made thereto without departing from the spirit of the invention.

It should also be understood that certain features of the invention, which are, for clarity, described in the context of separate embodiments, may be provided in combination in a single embodiment. Conversely, various features of the invention which are, for brevity, described in the context of a single embodiment, may also be provided separately or in any appropriate sub-combinations.

1. An ablation device including:
a needle with a needle body and a needle head, and
at least one positive temperature coefficient (PTC) member in a heat-transferrable relationship with said needle body.

2. A device according to claim 1 wherein said PTC member comprises a sleeve member surrounding at least part of said needle body.

3. A device according to claim 1 wherein said needle body is solid.

4. A device according to claim 1 further including an inner electrically insulating sleeve surrounding and in contact with at least part of an outer cylindrical surface of said needle body, an electrode sleeve surrounding and in contact with at least part of an outer cylindrical surface of said inner insulating sleeve, an outer electrically insulating sleeve surrounding and in contact with at least part of an outer cylindrical surface of said electrode sleeve, and an isolating electrically insulating layer surrounding and in contact with at least part of an outer cylindrical surface of said needle body.

5. A device according to claim 1 wherein said needle body and said needle head are integral with each other.

6. A device according to claim 4 wherein said isolating insulating layer is made at least principally of polytetrafluoroethylene.

7. A device according to claim 4 wherein said inner electrically insulating sleeve and/or said outer electrically insulating sleeve is made at least principally of polytetrafluoroethylene.

8. A device according to claim 1 wherein said needle body and/or said needle head is made at least principally of stainless steel.

9. A device according to claim 1 wherein the pointed end of said needle head is of an angle of between 10° to 20°.

10. A device according to claim 9 wherein the pointed end of said needle head is of an angle of substantially 14.5°.

11. A device according to claim 1 wherein said needle body is at least partly hollow and receives at least part of said PTC member.

12. A device according to claim 11 wherein said PTC member is in direct contact with said needle body.

13. A device according to claim 11 wherein said PTC member is at least partly hollow and receives at least part of an electrode.

14. A device according to claim 11 further including an outer electrically insulating sleeve surrounding and in contact with at least part of an outer cylindrical surface of said needle body.

15. A device according to claim 14 wherein said outer insulating sleeve is made at least principally of polytetrafluoroethylene.

16. A device according to claim 1 wherein said device is a unipolar ablation needle or a bipolar ablation needle.
